(19) United States
(12) Patent Application Publication
(10) Pub. No.: US 2013/0289465 A1
(43) Pub. Date: Oct. 31, 2013

(54) ADHESIVE PRODUCT WITH REMOVABLE TABS

(71) Applicant: James Hohnbaum, Glendale, AZ (US)

(72) Inventor: James Hohnbaum, Glendale, AZ (US)

(21) Appl. No.: 13/869,818

(22) Filed: Apr. 24, 2013

Related U.S. Application Data
(60) Provisional application No. 61/639,010, filed on Apr. 26, 2012.

(51) Int. Cl. A61F 13/02

(52) U.S. Cl. CPC A61F 13/024
USPC 602/57

(57) ABSTRACT

An adhesive product is disclosed, in which a portion of the adhesive product may be removable. The adhesive product may be an adhesive bandage. The adhesive bandage may comprise removable tabs. The removable tabs may allow a user to handle the adhesive bandage without contaminating the portion of the adhesive bandage that will be applied to a wound. The removable tabs may be non-adhesive, allowing a user to apply the adhesive bandage without the adhesive bandage adhering to the user's fingers. The removable tabs may be separated from the adhesive bandage once the adhesive bandage has been secured in place.

### CROSS-REFERENCE TO RELATED APPLICATIONS

This application claims priority to U.S. Provisional Application Ser. No. 61/639,010, entitled “ADHESIVE PRODUCT WITH REMOVABLE TABS,” and filed on Apr. 26, 2012 the contents of which are incorporated by reference herein in its entirety.

### BACKGROUND

Adhesive bandages are commonly used to protect wounds and increase the speed of healing. Adhesive bandages often comprise an adhesive portion and an absorbent pad. The adhesive portion adheres to the area around a wound and the absorbent pad covers the wound. Adhesive bandages often come with two overlapping strips which cover the adhesive portion. In order to apply the adhesive bandage, a user must remove the overlapping strips and grasp the adhesive portion of the adhesive bandage. By grasping the adhesive portion, a user introduces bacteria and other contaminants to the adhesive bandage, which may then contaminate the wound which the adhesive bandage is meant to protect. Additionally, as the adhesive bandage adheres to the user's fingers, the accuracy of bandage placement may be diminished.

As such, a need exists for an adhesive product which may be applied without contact with the adhesive portion of the bandage.

### SUMMARY

An adhesive product is disclosed, in which a portion of the adhesive product may be removable. The adhesive product may be an adhesive bandage. The adhesive bandage may comprise an adhesive portion and an absorbent pad. The adhesive portion may be adhesive on the bottom side. The adhesive bandage may comprise removable tabs. The removable tabs may be connected to ends of the adhesive portion. The removable tabs may be separated from the adhesive portion by perforations.

### BRIEF DESCRIPTION OF THE DRAWINGS

FIG. 1 illustrates a bottom view of an adhesive bandage according to various embodiments of the disclosure.

FIG. 2 illustrates a perspective view of an adhesive bandage according to various embodiments.

FIG. 3 illustrates a cross-section view of an adhesive bandage according to various embodiments.

FIG. 4 illustrates a cross-section view of an adhesive bandage according to various embodiments.

FIGS. 5a-5b illustrate bottom views of adhesive bandages according to various embodiments.

FIG. 6 illustrates a process for applying an adhesive bandage according to various embodiments.

### DETAILED DESCRIPTION

The detailed description of exemplary embodiments herein makes reference to the accompanying drawings and pictures, which show various embodiments by way of illustration. While these various embodiments are described in sufficient detail to enable those skilled in the art to practice the disclosure, it should be understood that other embodiments may be realized and that logical and mechanical changes may be made without departing from the spirit and scope of the disclosure. Thus, the detailed description herein is presented for purposes of illustration only and not of limitation. For example, the steps recited in any of the method or process descriptions may be executed in any order and are not limited to the order presented. Furthermore, any reference to singular includes plural embodiments, and any reference to more than one component may include a singular embodiment.

In the detailed description herein, references to “various embodiments,” “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. After reading the description, it will be apparent to one skilled in the relevant art(s) how to implement the disclosure in alternative embodiments.

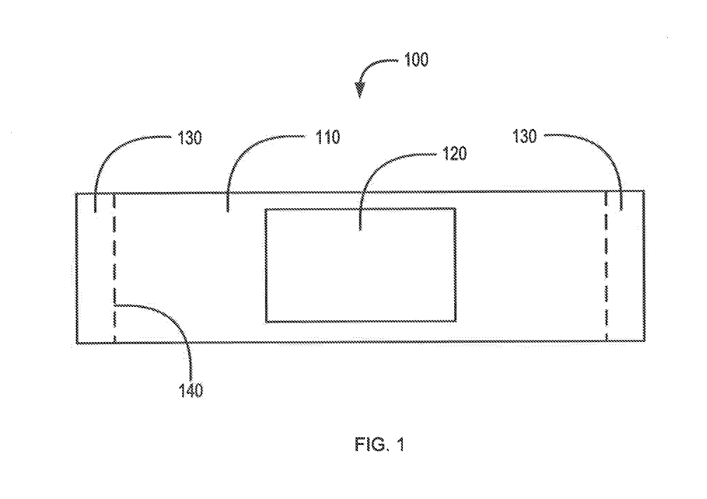

Referring to FIG. 1, a perspective view of an adhesive bandage 100 is illustrated according to various embodiments. Adhesive bandage 100 may comprise an adhesive portion 110, an absorbent pad 120, and removable tabs 130. In various embodiments, the adhesive portion 110 and the removable tabs 130 may be separated by perforations 140. The adhesive bandage 100 may be any size, shape, color, or design. For example, adhesive bandages are often flesh colored and ovoid. However, adhesive bandage 100 may be rectangular, square, “butterfly,” or any other shape, size or color.

Adhesive portion 110 may comprise any material capable of adhesive properties. For example, the material may include woven fabric, plastic or latex rubber which has an adhesive substance thereon. The adhesive portion 110 may comprise adhesive on a bottom side of the adhesive portion 110, and be non-adhesive on a top side of the adhesive portion 110. In various embodiments the adhesive portion 110 may have perspiration holes throughout, in order to allow moisture to pass through the adhesive bandage 100. In various embodiments the adhesive portion 110 may have adhesive properties itself without an additional adhesive substance thereon.

The adhesive bandage 100 may comprise an absorbent pad 120. The absorbent pad 120 may be located on the bottom side of the adhesive portion 110. In various embodiments the absorbent pad 120 may be located substantially at the center of the adhesive portion 110, however the absorbent pad 120 may be located at any position on the bottom side of the adhesive portion 110. The absorbent pad 120 may be a variety of shapes. In the illustrated embodiment, the absorbent pad 120 is rectangular. However, the absorbent pad 120 may be square, circular, ovoid, or any other shape capable of covering a wound. The absorbent pad 120 may be medicated with an antiseptic solution. Absorbent pad 120 may be surrounded by a porous-polymer coating such that the absorbent pad 120 does not stick to a wound.

The adhesive bandage 100 may comprise removable tabs 130. The removable tabs 130 may be connected to ends of the adhesive portion 110. The removable tabs 130 may be any shape. For example, the removable tabs 130 may be crescent shaped, rectangular, square, circular, triangular, linear, or any other shape which may be grasped by fingers. In various embodiments, the removable tabs 130 may be a different color that the adhesive portion 110. The different color may allow a user to identify which part of the adhesive bandage 100 is removable. However, the removable tabs 130 may be the same color as the adhesive portion 110. In various embodiments, the removable tabs 130 may be comprised substantially of the same material as the adhesive portion 110. However, in various embodiments the removable tabs 130 may comprise different materials than the adhesive portion 110. The removable tabs 130 may be connected to and/or a continuation of the adhesive portion 110.

The removable tabs 130 may be separated from the adhesive portion 110 by a series of perforations 140. The perforations 140 may comprise a series of holes through the material comprising the adhesive portion 110 and/or the removable tabs 130. The perforations 140 may be arranged linearly, allowing the material to be torn along the perforations 140. In response to a stress applied to the adhesive bandage 100, the material between the perforations may have a relatively weak tensile strength compared to other portions of the adhesive bandage 100, resulting in the removable tabs 130 tearing away from the adhesive portion 110. The removable tabs 130 may be separated by pulling on the removable tabs 130 once the adhesive bandage 100 has been secured in place over a wound, such that the perforations 140 tear and the removable tabs 130 are no longer connected to the adhesive portion 110.

In various embodiments, the adhesive bandage 100 may be scored in order to separate the removable tabs 130 from the adhesive portion 110. The score may comprise a groove or cut between the removable tabs 130 and the adhesive portion 110. Due to the scoring, the adhesive bandage 100 may have a smaller cross-section at the score compared to other portions of the adhesive bandage 100. Thus, in response to a stress applied to the adhesive bandage 100, the material along the score may have a relatively weak tensile strength compared to other portions of the adhesive bandage 100, resulting in the removable tabs 130 tearing away from the adhesive portion 110. Furthermore, the present disclosure is consistent with any structure or method which allows the removable tabs 130 to be separated from the adhesive portion 110.

In various embodiments, the removable tabs 130 may not comprise adhesive on the top or bottom sides of the removable tabs 130. Thus, a user applying the adhesive bandage 100 may handle the adhesive bandage 100 by the removable portions without the adhesive bandage 100 adhering to the user. Once the adhesive bandage 100 has been applied, the user may remove the removable tabs 130 and discard them.

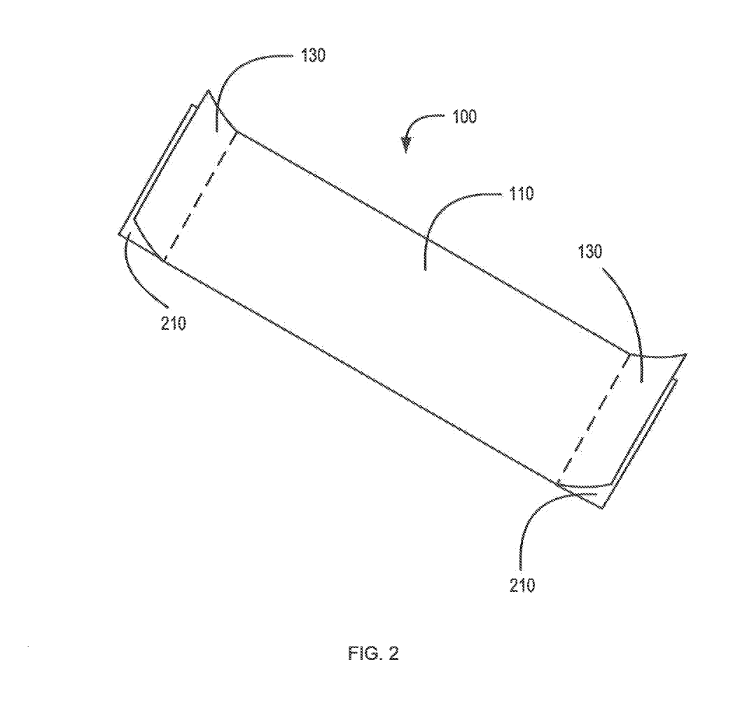

Referring to FIG. 2, a perspective view of an adhesive bandage 100 is illustrated according to various embodiments. The adhesive bandage 100 may comprise a covering 210. The covering 210 may adhere to the bottom side of the adhesive portion 110 in order to protect the adhesive portions 110 from contamination, as well as to maintain the adhesive properties of the adhesive portion 110. The covering 210 may be comprised of a variety of materials including but not limited to paper or plastic. In various embodiments the covering 210 may be non-adhesive, however the adhesive properties of the adhesive portion 110 may cause the covering 210 to adhere to the adhesive portion 110. In various embodiments, the covering 210 may be a single continuous piece of material. The covering 210 may be substantially the same shape as the adhesive portion 110 and the removable tabs 130. The covering 210 may be secured to the adhesive bandage 100 by the adhesive bottom side of the adhesive bandage 100. In embodiments where the removable tabs 130 are not adhesive on the bottom side, the removable tabs 130 may not adhere to the covering 210. Thus, a user may easily separate the removable tabs 130 from the covering 210 and peel the covering 210 from the adhesive bandage 100, such that the adhesive bandage 100 may be applied to a wound.

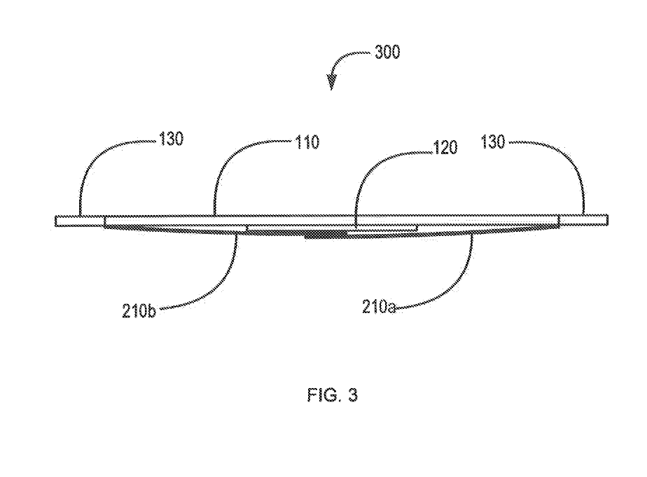

Referring to FIG. 3, a cross-section view of an adhesive bandage 300 is illustrated according to various embodiments. The removable tabs 130 may extend beyond the covering 210. The covering 210 may comprise a plurality of portions, such as first portion 210a and second portion 210b. Covering 210 may not need to cover the removable tabs 130, because the removable tabs 130 may not contain adhesive properties which would need to be protected by a covering. The first portion 210a of the covering 210 may overlap the second portion 210b, such that a user may grasp the overlapping portion of the first portion 210a and remove it from the adhesive bandage 300. The user may then remove the second portion 210b. After securing the adhesive bandage 300 in place using the removable tabs 130, the user may tear away the removable tabs 130.

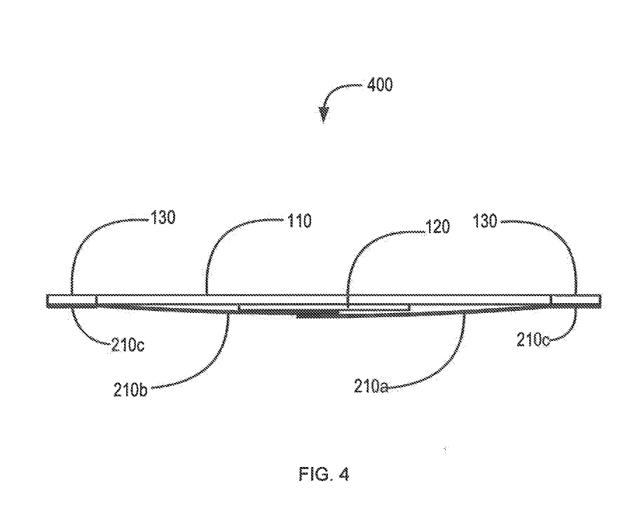

Referring to FIG. 4, a cross-section view of an adhesive bandage 400 is illustrated according to various embodiments. In the embodiment of FIG. 4, the removable tabs 130 may be adhesive or contain adhesive on the bottom side. The covering 210 may comprise a plurality of portions. First portion 210a and second portion 210b may be similar to those described with reference to FIG. 3. Tab cover portions 210c may cover the removable tabs 130. The tab cover portions 210c may adhere to the removable tabs 130 via the adhesive properties of removable tabs 130. In various embodiments, the tab cover portions 210c are disconnected from first portion 210a and second portion 210b. However, the tab cover portions 210c may be connected to the first portion 210a and the second portion 210b, wherein perforations separate the tab cover portions 210c from the first portion 210a and the second portion 210b. In various embodiments consistent with FIG. 4, a user may remove the first portion 210a and the second portion 210b. The tab cover portions 210c may remain attached to the removable tabs 130. The user may apply the adhesive bandage 100, by grasping the removable tabs 130 and the tab cover portions 210c. The user may tear away the removable tabs 130 and the tab cover portions 210e after applying the adhesive bandage 100. In such embodiments, the adhesive portions 110 and the removable tabs 130 may be comprised of the same material and contain the same adhesive properties on the bottom.

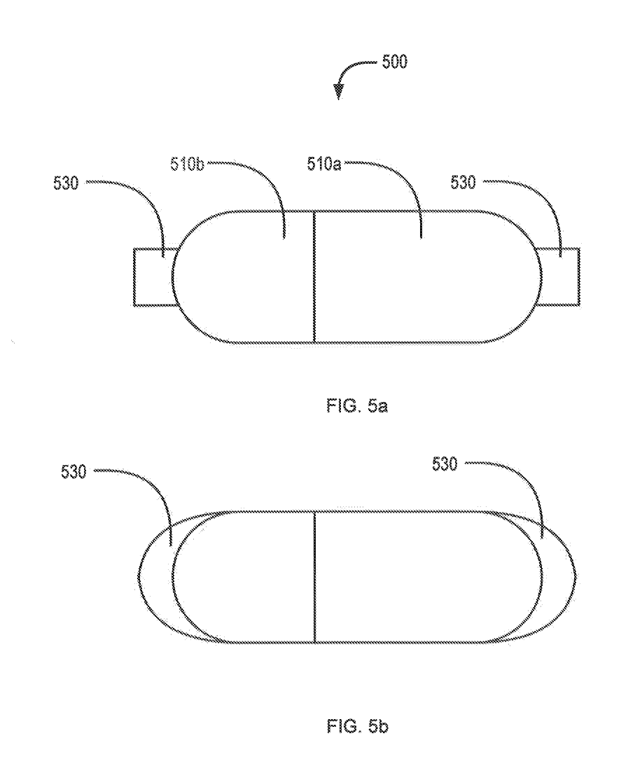

Referring to FIG. 5a, a bottom view of a bandage 500 is illustrated according to various embodiments. The bandage 500 may have rounded ends. A covering may comprise first portion 510a and second portion 510b. First portion 510a may overlap second portion 510b. The covering may adhere to the adhesive portion of the bandage 500. The bandage 500 may comprise removable tabs 530. The removable tabs 530 may be non-adhesive. The removable tabs 530 may be separated from the adhesive portion by perforations.

Referring to FIG. 5b, a bottom view of a bandage 500 is illustrated according to various embodiments. In the illustrated embodiment, the removable tabs 530 may be crescent shaped.

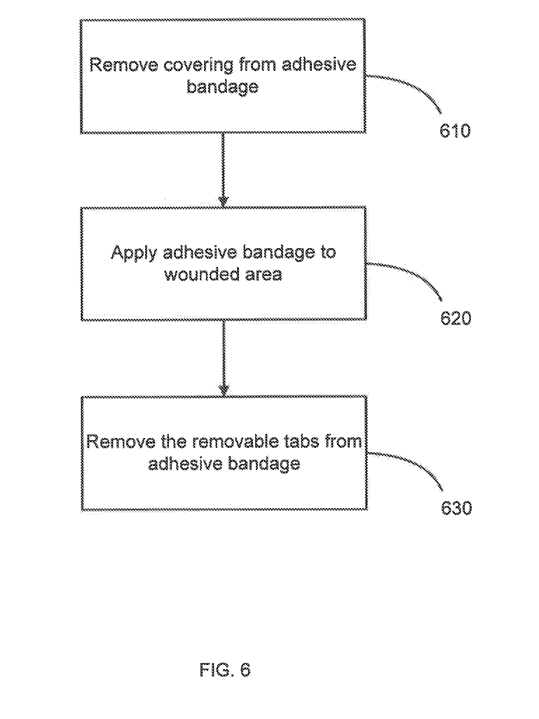

Referring to FIG. 6, a flowchart is illustrated describing a method of applying an adhesive bandage with removable tabs according to various embodiments. In step 610, a user may remove a covering from the adhesive bandage. The user may grasp a non-adhesive removable tab in one hand and the covering in the other hand. The user may pull the removable tab and the covering apart, separating the covering from the adhesive bandage. In various embodiments, the covering may comprise multiple portions. The user may remove the portions that cover the adhesive portions. Any covering portions that adhere to the removable tabs may remain attached to the removable tabs.

In step 620, the user may apply the adhesive bandage to a wounded area. The user may grasp the removable tab on each end of the adhesive bandage. The user may place an absorbent pad over the wound, and press on the top side of the adhesive portion of the adhesive bandage. By grasping the adhesive bandage by the removable tabs, the user may prevent bacteria and other contaminants from contaminating the adhesive portion of the adhesive bandage. Furthermore the user may more accurately place the adhesive bandage and prevent the adhesive bandage from sticking to the user's fingers.

In step 630, the user may remove the removable tabs from the adhesive bandage. The user may grasp the removable tabs and pull them in a direction away from the adhesive portions. Due to the pulling force applied by the user, and the adhesive force between the adhesive portions and the wounded area, the perforations between the removable tabs and the adhesive portions may tear. Thus, the removable tabs may no longer be attached to the adhesive bandage, and the adhesive bandage may be accurately and securely placed over a wound.

Although adhesive products have been disclosed herein with reference to adhesive bandages, practitioners skilled in the art will appreciate that the adhesive products may comprise any adhesive product with removable tabs. For example, the present disclosure contemplates labels, wherein the label comprises an adhesive portion and non-adhesive removable tabs, such that a user may handle the non-adhesive removable tabs in order to separate the label from a backing sheet and apply the label to a desired location. The user may then tear the removable tabs from the label. The present disclosure also contemplates similar embodiments with removable tabs related to stickers, license plate tabs, name tags, ski lift tickets, or any other product containing an adhesive.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. Reference to an element in the singular is not intended to mean “one and only one” unless explicitly so stated, but rather “one or more.” Moreover, where a phrase similar to ‘at least one of A, B, and C.’ or ‘at least one of A, B, or C’ is used in the specification, it is intended that the phrase be interpreted to mean that. A alone may be present in an embodiment, B alone may be present in an embodiment, C alone may be present in an embodiment, or that any combination of the elements A, B and C may be present in a single embodiment; for example, A and B, A and C, B and C, A and B and C.

All structural, chemical, and functional equivalents to the elements of the above-described exemplary embodiments that are known to those of ordinary skill in the art are expressly incorporated herein by reference and are intended to be encompassed by the present disclosure. Moreover, it is not necessary for a device or method to address each and every problem sought to be solved by the present disclosure, for it to be encompassed by the present claims. Furthermore, no element, component, or method step in the present disclosure is intended to be dedicated to the public. No element herein is to be construed under the provisions of 35 U.S.C. 112, sixth paragraph, unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

1. An adhesive device comprising:
an adhesive portion;
an absorbent pad coupled to the adhesive portion;
a removable tab coupled to the adhesive portion; and
a plurality of perforations separating the adhesive portion and the removable tab.

2. The adhesive device of claim 1, wherein the removable tab is non-adhesive.

3. The adhesive device of claim 1, wherein the removable tab is configured to tear away from the adhesive portion at the plurality of perforations.

4. The adhesive device of claim 1, further comprising a covering coupled to the adhesive portion.

5. The adhesive device of claim 4, wherein the covering is substantially the same shape as the adhesive portion and the removable tab.

6. The adhesive device of claim 4, wherein the covering is substantially the same shape as the adhesive portion.

7. The adhesive device of claim 4, wherein the covering comprises a first portion, a second portion overlapping the first portion, and a tab cover portion, wherein the removable tab is adhesive, and wherein the tab cover portion is coupled to the removable tab.

8. The adhesive device of claim 7, wherein the tab cover portion is separated by at least one of the first portion and the second portion by a plurality of perforations.

9. The adhesive device of claim 1, wherein the removable tab is crescent shaped.

10. A bandage comprising:
an adhesive portion comprising a non-adhesive top side and an adhesive bottom side;
an absorbent pad coupled to the bottom side of the adhesive portion, wherein the absorbent pad is located substantially at a center of the adhesive portion;
a first removable tab coupled to a first end of the adhesive portion;
a second removable tab coupled to a second end of the adhesive portion, wherein the first removable tab and the second removable tab are comprised of substantially the same material as the adhesive portion;
a first plurality of perforations separating the first removable tab and the second removable tab from the adhesive portion; and
a non-adhesive covering coupled to the bottom side of the adhesive portion.

11. The bandage of claim 10, wherein the covering consists of a single portion, wherein the covering is substantially the same shape as the adhesive portion, the first removable tab, and the second removable tab, and wherein the covering does not adhere to the first removable tab or the second removable tab.

12. The bandage of claim 10, wherein the covering comprises a first portion coupled to the adhesive portion, and a second portion coupled to the adhesive portion and overlapping the first portion.

13. The bandage of claim 12, wherein the first removable tab and the second removable tab comprise an adhesive bottom side, and wherein the covering further comprises a first tab cover portion coupled to the first removable tab and a second tab cover portion coupled to the second removable tab.

14. The bandage of claim 13, wherein the first tab cover portion and the second tab cover portion are discontinuous from the first portion and the second portion.

15. The bandage of claim 13, wherein the first tab cover portion and the second tab cover portion are separated from the first portion and the second portion by a second plurality of perforations.

16. The bandage of claim 10, wherein the first removable tab and the second removable tab are configured to tear away from the adhesive portion.

17. The bandage of claim 10, wherein the first removable tab and the second removable tab are a different color than the adhesive portion.

18. A method of applying a bandage comprising:
removing a covering from an adhesive portion of the bandage;
grasping as removable tab of the bandage;
securing the adhesive portion of the bandage adjacent to a wound; and
separating the removable tab from the bandage.

19. The method of claim 18, wherein the separating the removable tab comprises tearing a plurality of perforations coupling the removable tab to the adhesive portion.

20. The method of claim 18, wherein the covering consists of as single piece of material, and wherein the removing comprises grasping the removable tab and the covering, and pulling the covering away from the bandage.
